(19) United States
(12) Patent Application Publication
(10) Pub. No.: US 2018/0110462 A1
(43) Pub. Date: Apr. 26, 2018

(54) DEVICE, SYSTEM AND METHOD FOR DETECTING ILLNESS- AND/OR THERAPY-RELATED FATIGUE OF A PERSON

(71) Applicant: KONINKLIJKE PHILIPS N.V., EINDHOVEN (NL)

(72) Inventors: SIMA ASVADI, EINDHOVEN (NL); KIRAN HAMILTON J. DELLIMORE, EINDHOVEN (NL); VANDA LUCIA DE CARVALHO VITTORINO DE ALMEIDA, VELDHOVEN (NL); RONALDUS MARIA AARTS, GELDROP (NL); MARC ANDRE DE SAMBER, LOMMEL (BE)

(21) Appl. No.: 15/564,990

(22) PCT Filed: Apr. 5, 2016
(86) PCT No.: PCT/EP2016/057383

§ 371 Date: Oct. 6, 2017

(30) Foreign Application Priority Data
Apr. 9, 2015 (EP) 15162993.8

(51) Int. Cl. A61B 5/00; A61B 5/145; A61B 5/11; A61B 5/16; A61B 5/0205; G16H 10/60

(52) U.S. Cl. CPC A61B 5/4815; A61B 5/14546; A61B 5/1118; A61B 5/4809; A61B 5/4803; A61B 5/1116; A61B 5/0533; A61B 5/7282; A61B 5/0205; A61B 5/4866; A61B 5/4884; G16H 10/60; A61B 5/4857; A61B 5/163

(57) ABSTRACT

The present invention relates to a device, system and method for detecting illness- and/or therapy-related fatigue of a person in an easy and reliable way. For this purpose the device comprises an input unit (20) for obtaining white blood cell count data related to the person's white blood cell count, hemoglobin level data related to the person's hemoglobin level and cortisol level data related to the person's cortisol level, and an analyzer (21) for detecting illness- and/or therapy-related fatigue of the person based on the obtained white blood cell count data, hemoglobin level data and cortisol level data.

### FIELD OF THE INVENTION

The present invention relates to a device, system and method for detecting illness- and/or therapy-related fatigue of a person.

### BACKGROUND OF THE INVENTION

Various types of disease like cancer, chronic inflammatory conditions (e.g. rheumatoid arthritis, inflammatory multiple sclerosis) and non-inflammatory conditions like Parkinson's disease and/or the treatment for such types of disease may cause illness-related fatigue in patients. Illness-related fatigue affects a patient's condition (both physically and mentally) and, consequently, impacts the subsequent development of the illness as well as subsequent treatment.

Cancer-related fatigue (CRF) is a highly prevalent and debilitating symptom experienced by most cancer patients during, and often for considerable periods after, radiotherapy/chemotherapy treatment. CRF affects the physical, mental, and emotional capacity of the patients, and hence has a major influence on their quality of life. CRF is described as a subjective feeling of tiredness, weakness, or lack of energy that influences daily activities and quality of life. In healthy people fatigue has a protective function in response to physical or physiological stress. In cancer patients however, fatigue has lost its function and does not diminish with rest.

Furthermore, it is the common practice that patients who receive chemotherapy treatment make an assessment of their own “well-being” and use that judgment as the basis for seeking medical attention in the case of potentially life threatening risk of neutropenia.

Fatigue is often reported as the effect of both cancer tumor activities and cancer treatment routines such as chemotherapy or radiation therapy. This is often superimposed on the physiological and psychological stress that is involved in dealing with cancer which is demonstrated by the fact that individuals with cancer are disproportionately affected by various circadian rhythm disorders, e.g., sleep disturbance and insomnia, relative to the general population.

This combined effect forms the core of the problem demonstrated by the fact that only a fraction of all chemotherapy patients who visit the hospital actually are in need of hospitalization. This creates a burden on the healthcare system in many countries due to the expense of unnecessary hospitalizations on the one hand and at the same time contributes to resource limitation/shortage caused by the need to accommodate cancer patients during these hospitalizations resulting in under-treatment of another group of patients.

During chemotherapy or other cancer related treatments such as radiotherapy periods it is of crucial importance to have a view on the overall condition of the patients as defined by general health and wellbeing in combination with the therapeutic (and side) effect of the specific therapy (medication). This is particularly essential in order to follow up the patient's status and manage their treatment, as well as to prevent expensive and unnecessary hospitalizations.

Several strategies are currently used to assess the fatigue state of cancer patients; however, they are in most cases not objective. This may be due in large part to the definition of CRF in the clinical guidelines for cancer therapy management as a subjective symptom. As a result, many subjective methods have been developed to assess the state of a cancer patient's well-being, including 43 self-assessment questionnaires available in English (with 55 different names, e.g., the BFI scale, Brief Fatigue Inventory; EORTC QLQ C30 FS, European Organisation for Research and Treatment of Cancer Quality of Life Questionnaire Fatigue subscale; FSS, Fatigue Severity Scale; FACT F, Functional Assessment of Cancer Therapy Fatigue subscale; POMS F, Profile of Mood States Fatigue subscale, etc.). The results of these self-assessments are typically combined with a physiological evaluation by a doctor (e.g. at-point-of-care), in order to develop an assessment of the cancer patient's fatigue state. However, this approach is ad hoc and does not permit CRF to be continuously measured. Nevertheless, in recent years there has been increasing interest in a more objective measure of fatigue in cancer patients, using actigraphy. Several studies have been performed, including one which reported that individuals with marked rest/activity rhythms had better quality of life and reported significantly less fatigue during cancer therapy.

Adequate treatment of CRF starts with identifying the contributing factors and forming a CRF history covering its severity, pattern, contributing and relieving factors, as well as the impact that they have on day to day activities. Relying on the data provided by the patients is subject to significant fluctuations and errors originating from the very source that is under investigation in dealing with fatigue monitoring. This remains a major challenge despite the fact that significant attempts have been made to standardize the data obtaining processes (through fatigue guidelines) and tools (questionnaires). Furthermore, such subjective methods are only beneficial after the onset of fatigue and have no predictive thus preventive value.

U.S. Pat. No. 8,639,639 discloses a method, system and apparatus related to predicting possible outcomes in a multi-factored disease, disorder or condition. The method comprises receiving an input, the input representative of one or more diagnostic factors of a multi-factored disease, disorder or condition, and predicting a possible outcome based on the input, wherein predicting a possible outcome based on the input comprises constructing a classification tree of two or more diagnostic factors of the multi-factored disease, disorder or condition and performing discriminant analysis of the two or more diagnostic factors.

Gerber L H, Stout N, McGarvey C, et al., Factors predicting clinically significant fatigue in women following treatment for primary breast cancer, Supportive Care in Cancer, 2011; 19(10):1581-1591, discloses an assessment of a number of variables in women newly diagnosed with primary breast cancer (BrCa) to determine whether biological and/or functional measures are likely to be associated with the development of clinically significant fatigue (CSF). Objective measures and descriptive variables included history, physical examination, limb volume, hemoglobin, white blood cell count, and glucose.

### SUMMARY OF THE INVENTION

It is an object of the present invention to provide an improved device, system and method for objectively and early detecting illness- and/or therapy-related fatigue of a person.

In a first aspect of the present invention a device for detecting illness- and/or therapy-related fatigue of a person is presented, said device comprising
an input unit for obtaining white blood cell count data related to the person's white blood cell count, hemoglobin level data related to the person's hemoglobin level and cortisol level data related to the person's cortisol level, and
an analyzer for detecting illness- and/or therapy-related fatigue of the person based on the obtained white blood cell count data, hemoglobin level data and cortisol level data.

In a further aspect of the present invention a corresponding method is presented.

In yet a further aspect of the present invention a system for detecting illness- and/or therapy-related fatigue of a person is presented, said system comprising:
a white blood cell counter for counting the white blood cells of the person,
a hemoglobin level sensor for determining the hemoglobin level of the person, a cortisol level sensor for determining the cortisol level of the person,
a device as disclosed herein for detecting illness- and/or therapy-related fatigue of the person based on the data obtained from the white blood cell counter, the hemoglobin level sensor and the cortisol level sensor.

In yet further aspects of the present invention, there are provided a computer program which comprises program code means for causing a computer to perform the steps of the method disclosed herein when said computer program is carried out on a computer as well as a non-transitory computer-readable recording medium that stores therein a computer program product, which, when executed by a processor, causes the method disclosed herein to be performed.

Preferred embodiments of the invention are defined in the dependent claims. It shall be understood that the claimed method, system, computer program and medium have similar and/or identical preferred embodiments as the claimed system and as defined in the dependent claims.

According to the present invention at least the three main parameters including the person's white blood cell count, the person's hemoglobin level and the person's cortisol level are used to objectively assess illness-/therapy-related fatigue. This helps assessing the person's condition, e.g. while the person is undergoing a therapy. Based on such an objective assessment the person's treatment can be adapted. Further, it enables more effective timing of treatment interventions and, consequently, increases the patient's well-being. Still further, continuous or semi-continuous monitoring of the person is possible, accounting for both short term (i.e., hour to hour, day to day) and long term (week to week, month to month) fluctuations and variations in fatigue parameters.

Using at least the proposed parameters a person can follow his status after every therapy session, e.g. after each chemotherapy/radiotherapy session, with the aim to predict, recognize and hence relieve the side effects. This approach can also detect any abnormality in the recovery progress resulting in a significant reduction of the negative impact of the treatment on person's life.

In an embodiment said analyzer is configured to monitor trends over time in at least one, preferably all, of the obtained white blood cell count data, hemoglobin level data and cortisol level data. By monitoring the trends early detection of fatigue is possible.

In another embodiment the device further comprises an interface for issuing fatigue information, user information, therapy recommendations and/or decision support if fatigue is detected. Fatigue information may include information if fatigue and to which extent fatigue has been detected, e.g. informing the person that the fatigue is an expected side effect of the therapy. User information may include information, e.g. for a doctor, informing the user about the fatigue, e.g. about a level of fatigue as detected over time. Therapy recommendations may include recommendations for the person and/or a user how the therapy should be continued or modified or which other therapies should be applied, for instance in order to reduce the level of fatigue. Decision support may include information e.g. for a doctor supporting him to make decisions with respect to the person, e.g. how to continue with the therapy.

In an embodiment said analyzer is configured to determine a fatigue level and for monitoring the fatigue level over time. This enables an early recognition if the person suffers from fatigue. The fatigue level may e.g. be determined by providing a score for each parameter used in the detection of fatigue and for commonly evaluating the different scores to obtain a combined score reflecting or representing the fatigue level.

Also in this embodiment the device may further comprise an interface for issuing fatigue information if a fatigue level above a predetermined and/or person-related fatigue level threshold is detected. The fatigue level threshold may be a general threshold, but may alternatively be adapted to the respective person, e.g. based on type of illness and/or therapy as well as personal features of the person, such as age, weight and height, gender, health status and record, genetic predisposition, etc.

Preferably, said analyzer is configured to additionally use chronobiology information related to the chronobiology of the person for detecting fatigue. It has been found that parameters of the person's chronobiology are related to illness or treatment related fatigue. For instance, the circadian rhythm representing the influence of chronotoxicity of cancer treatment, which is determined by the biological clock of the patient at the cell level, is related to fatigue. Monitoring of the three above mentioned parameters over time can provide the required information on the patient-specific chronobiology aspects of fatigue level and/or treatment program. Other parameters include sleep disorders, muscle fatigue, heart rate, temperature, rest-activity and cortisol/melatonin secretion. These parameters can be measured by various sensors or can be collected by available devices used by the person, such as a smartphone, smart watch, smart patch, a camera, etc. Thus, the use of chronobiology information and the link with fatigue further improves the correct, early and objective detection of fatigue. Further, the rate of recovery of the person may be predicted.

In still another embodiment said input unit is configured to obtain person activity data (also called “soft data” herein) related to one or more activities of the person, wherein said analyzer is configured to additionally use the obtained person activity data for detecting fatigue. Said person activity data may include one or more of diet or eating habits, exercise frequency, activity level, sleep disturbance (e.g., through activity based e.g. restless motion at night, or brain signal e.g. delta wave measurement, etc.), speech pattern, eye movement and body posture. The proposed system thus may comprise additional corresponding sensors or means for acquiring the respective data. These sensors might be ‘embedded’ or otherwise connected (wired, wireless, via the cloud, etc.) to the system.

The input unit may also be configured to obtain physiological data (also called “hard data” herein) related to one or more physiological parameters of the person, wherein said analyzer is configured to additionally use the obtained physiological data for detecting fatigue. Said physiological data may include one or more of biomarker data from blood or other biomaterials such as saliva, urine, tear fluid or hair, melatonin concentration, red blood cell count (to indicate anemia), anti-oxidant concentration in blood, vital sign measurements such as blood pressure, heart rate, respiratory rate or skin conductance. Also for acquisition of the respective data the proposed system may comprise corresponding sensors or means. Thus, in these embodiments the problems of the known methods and devices are solved by multi-component monitoring of illness- or therapy related fatigue parameters.

In still another embodiment said analyzer is configured to determine for the obtained data the respective deviation from a predetermined range, in particular a person-related range, for combining, in particular adding, said deviations and for detecting fatigue, in particular a fatigue level, based on the combined deviations. The combination may be obtained by various multi-criteria decision analysis techniques, e.g., weighted summation, weighted product model, aggregated indices randomization method, etc.

### BRIEF DESCRIPTION OF THE DRAWINGS

These and other aspects of the invention will be apparent from and elucidated with reference to the embodiment(s) described hereinafter. In the following drawings

FIG. 1 shows a schematic diagram of a first embodiment of a system and a device according to the present invention,

FIG. 2 shows a schematic diagram of a second embodiment of a system and a device according to the present invention,

FIG. 3 shows a schematic diagram of a third embodiment of a system and a device according to the present invention,

FIG. 4 shows a diagram illustrating the interrelation of biomarkers determining fatigue level, and

FIGS. 5 to 12 show graphs of various parameters illustrating the normal course and the influence of illness and/or therapy.

### DETAILED DESCRIPTION OF THE INVENTION

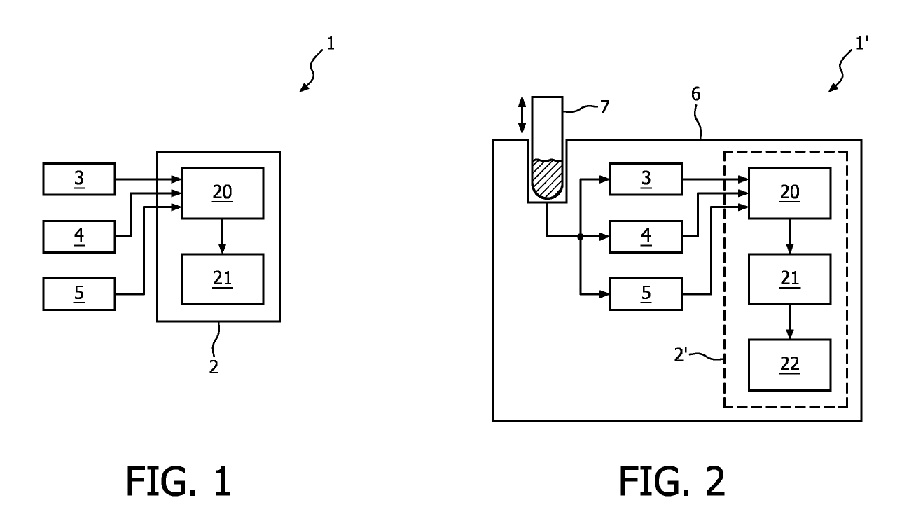

FIG. 1 shows a schematic diagram of a first embodiment of a system 1 and a device 2 according to the present invention for detecting illness- and/or therapy-related fatigue of a person. Besides the device 2, the system 1 comprises a white blood cell counter 3 for counting the white blood cells of the person, a hemoglobin level sensor 4 for determining the hemoglobin level of the person and a cortisol level sensor 5 for determining the cortisol level of the person. Based on the data obtained from the white blood cell counter 3, the hemoglobin level sensor 4 and the cortisol level sensor 5 the device detects if the person suffers from an illness- and/or therapy-related fatigue or is an early stage of such a fatigue. Further, the level of fatigue of the person may be monitored over time.

The device 2 comprises an input unit 20 for obtaining white blood cell count data related to the person's white blood cell count, hemoglobin level data related to the person's hemoglobin level and cortisol level data related to the person's cortisol level. An analyzer 21 processes the obtained white blood cell count data, hemoglobin level data and cortisol level data input data of the person and detects illness- and/or therapy-related fatigue of the person. The input data may be any kind of data interface which directly obtains the data from the respective sensor (i.e. the sensors 3-5 of the system), e.g. through a wireless or wired connection, to process the data on the fly and immediately detect if the person suffers from fatigue or is in an early stage. Alternatively, the data may be stored or buffered, e.g. in a storage medium, a hospital's data base, etc. for later processing by the device 2.

The analyzer 21 may be a processor of a separate device or a computer that is particularly programmed for carrying out the analysis.

The white blood cell counter 3 and the hemoglobin level sensor 4 may be sensors that count blood cells in a blood probe taken from the person using existing methods suitable for ex-vivo blood analysis. Alternatively, a variety of non-invasive blood counts may be performed, the majority of which allows in-vivo testing. These methods include electrical approaches (as e.g. described in Electrical admittance cuff for noninvasive and simultaneous measurement of haematocrit, arterial pressure and elasticity using volume-oscillometric method, Yamakoshi K l, Tanaka S, Shimazu H., Med. Biol>Eng. Comput. 1994 July; 32(4 Suppl):S99-107) or ultrasound approaches (as e.g. described in Noninvasive in vivo measurements of hematocrit. Secomski W et al., J. Ultrasound Med. 2003 April; 22(4):375-84) as well as a variety of known optical measurements of the white blood or red blood cell counting methods with or without labeling techniques (as e.g. described in Direct measurement of microvessel hematocrit, red cell flux, velocity, and transit time, Sarelius I H, Duling B R. Am J. Physiol, 1982, December; 243(6):H1018-26 and Noninvasive imaging of flowing blood cells using label free spectrally encoded flow cytometry, Lior Golan et al., Biomed Opt Express, 2012, June 1, 3(6) 1455-1464).

The cortisol level sensor 5 may be a sensor that senses the cortisol level in a blood, saliva, tear fluid, hair follicle, hair shaft, dermal tissue or urine probe of the person.

The system 1 may be used for performing detections of fatigues from time to time or for regularly or even continuously, e.g. to monitor trends over time. For this purpose, the analyzer 21 may be configured to monitor trends over time in at least one, preferably all, of the obtained white blood cell count data, hemoglobin level data and cortisol level data.

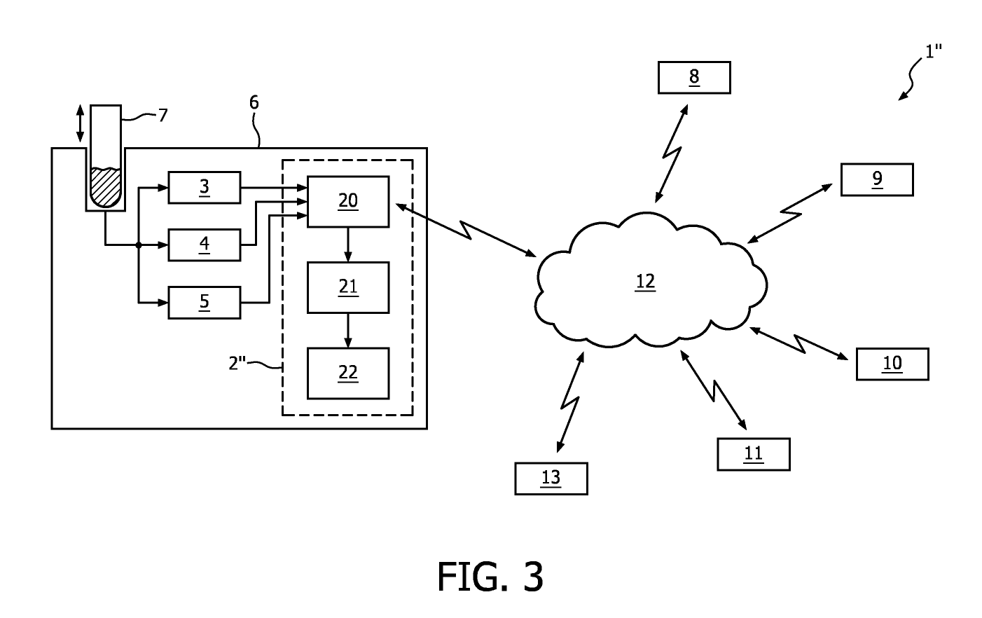

FIG. 2 shows a schematic diagram of a second embodiment of a system 1′ and a device 2′ according to the present invention. In this embodiment all elements of the system 1′ are integrated into a single apparatus 6, which may be a stationary or mobile apparatus, e.g. an apparatus which may be worn by the person or which may be used by a physician for visits of patients.

In an embodiment the apparatus 6 may be of the same or similar type as the device for home monitoring of hematological parameters of patients as described in WO 2014/024176 A1 or of the same or similar type as the commercial device Minicare H-2000, which is a remote monitoring system for patients undergoing chemotherapy. One or more probes 7 of a body fluid, particularly blood, are used for acquiring white blood cell count data, hemoglobin level data and cortisol level data. For this purpose the appropriate sensors 3, 4, 5 are incorporated into the apparatus 6 so that the person can perform a self-diagnosis.

The device 2′ further comprises an interface 22 for issuing fatigue information, user information, therapy recommendations and/or decision support if fatigue is detected. Fatigue information may include the determined fatigue level, a trend of the fatigue level over time, or information if fatigue is detected or not. User information may include information how and/or when to use the system 1′, or information that fatigue is detected, but that the detected level of fatigues is expected and “normal” for the kind of therapy which the person is undergoing. Therapy recommendations may include recommendations if and how the therapy of the person shall be continued, changed or stopped, e.g. if a chemotherapy shall be modified. Decision support may include information directed to a physician about the detected fatigue and supporting the physician to make a decision if and how the therapy shall be modified.

The analyzer 21 may further be configured to determine a fatigue level and for monitoring the fatigue level over time, wherein the interface 22 may be configured for issuing fatigue information if a fatigue level above a predetermined and/or person-related fatigue level threshold is detected.

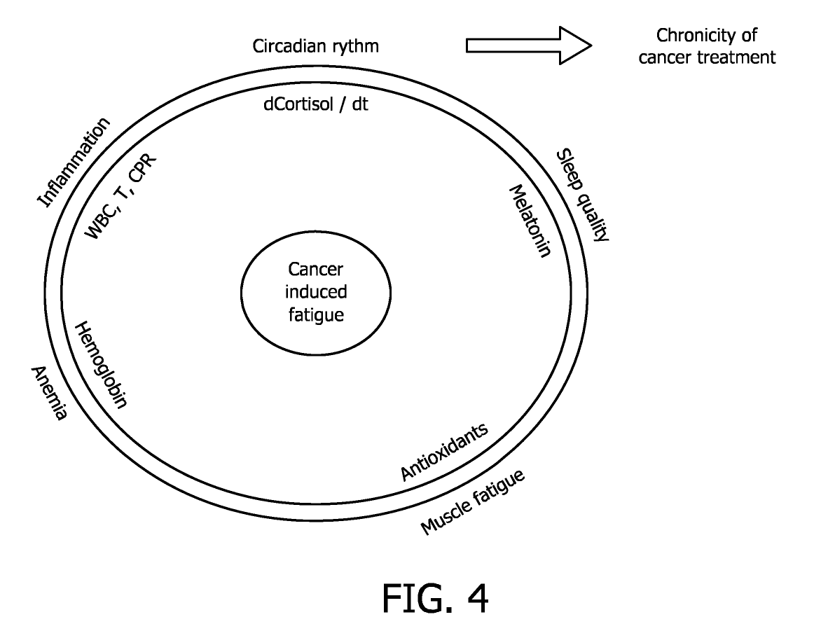

FIG. 3 shows a schematic diagram of a third embodiment of a system 1″ and a device 2″ according to the present invention. The device 2″ is preferably configured in the same way as the device 2′ shown in FIG. 2 and is incorporated into an apparatus 6 together with sensors 3, 4, 5. In another embodiment, however, the device 2″ is configured in the same way as the device 2 shown in FIG. 1 with external sensors 3, 4, 5.

The system 1″ employs a multi-component approach, combining “soft” data, which are particularly person activity data related to one or more activities of the person, and/or “hard” data, which are particularly physiological data related to one or more physiological parameters of the person, that are relevant to fatigue. The input unit 20 of the device 2″ is thus configured to obtain such person activity data and/or physiological data, and the analyzer 21 is configured to additionally use the obtained person activity data and/or physiological data for detecting fatigue. For instance, a semi-continuous assessment of the fatigue state of a cancer patient can thus be realized via a cancer-related fatigue (CRF) severity score reflecting the fatigue level.

The person activity data may include one or more of eating habits, exercise frequency, activity level, sleep disturbance, speech pattern, eye movement and body posture, and the physiological data may include one or more of biomarker data from blood or other biomaterials such as saliva, urine, tear fluid or hair, melatonin concentration, red blood cell count, anti-oxidant concentration in blood, vital sign measurements such as blood pressure, heart rate, respiratory rate or skin conductance. To obtain such additional sensors are used, as shown in FIG. 3, including one or more of a microphone 8, a video camera 9, other stationary and/or wearable sensors 10 (e.g. a vital signs camera, smart bed, smart chair, etc.) and wearable devices 11 (e.g. smart watches, smart phones, Google Glass, smart patches, electrode skull caps, etc.).

The wearable and non-wearable devices can be linked to device 2″ directly, via a wired or wireless network (e.g. a WiFi network), via the cloud 12, or simply via a telehub component. It may thus also be possible to control one or more of the various sensors 3-5, 8-11 when, how long and how often sensor data shall be acquired and provided to the device 2″.

A database 13 containing the patient's history as well as data from prior therapy cycles or from before starting the therapy may also be linked to the device 2″, e.g. also via the cloud 12. The fatigue level (e.g. as reflected by a CRF severity score) may be used in combination with the database 13 to manage the cancer patient's therapy by devising personalized exercise routines, nutritional advice and relaxation therapy. In addition, the fatigue level can be used by the physician to help in the scheduling of the next round of chemotherapy and to advise on hospital admission.

More specifically, the “soft” data (person activity data) can be obtained using the proposed device, preferably with an additional microphone and video camera, as well as with other wearable and/non-wearable devices, in the following ways:

1. Eating frequency—Under- or over-consumption of food may lead to fatigue. This can be measured using an accelerometer in a smart watch, along with an activity/motion classifier to identify the hand to head motion associated with eating and drinking.

2. Exercise/intense physical activity frequency—Low levels of intense physical activity and exercise are associated with increased fatigue. This can be measured using accelerometers in wearable devices (e.g., smart patch, smart watch, smart phone or Google Glass), along with a motion classification algorithm.

3. Eye Jitter—A fatigued patient will have ocular drift, ocular microtremors and microsaccades which are different from an unfatigued patient due to their inability to maintain visual fixation. This can be measured using a camera and an eye motion classification algorithm.

4. Indecision time—A more fatigued patient will take a longer time to complete the questionnaire because of impaired focusing and mental fog. Thus, fatigue will affect the time it takes for the patient to read the questionnaire and to input their response. The read and input time can be used as a sign of fatigue. This can be measured using a timer on device.

5. Postural sway—A fatigued patient will have increased postural instability. Fatigue influences postural control in the body in various ways. Physical fatigue due to muscular tiredness has been shown to influence the peripheral proprioceptive system, the central processing of proprioception as well as the force generating capacity of the neuromuscular system, which controls the motor impulses that make postural adjustments. Mental fatigue, on the other hand, impairs the peripheral proprioceptive system and the central processing of proprioception, which throws off the input and feedback to the neuromuscular system and results in decreased postural stability. This can be measured using a camera and/or a built-in accelerometer on a wearable device such as a smart phone, smart patch, Google Glass or smart watch, along with a motion classifier, to distinguish swaying from other motion.

6. Resting eye movements—A fatigued individual will have droopy eyelids and will blink more frequently than an un-fatigued individual. A camera built-in to the device along with a blinking classification algorithm can be used to measure the blinking.

7. Sleep disturbance—Delta waves are associated with deep sleep. By monitoring delta wave formation during sleep using an EEG skull cap, and a classification algorithm it is possible to identify depth of sleep, and determine if the cancer patient has had restful sleep. Sleep disturbance may also be measured using the accelerometer of the smart watch or smart patch device and an activity/motion classifier.

8. Speaking frequency of negative or tired words—When an individual is tired or stressed they often use more negative words or words associated with tiredness, e.g., “I am xxx”—exhausted, tired, stressed, etc. This can be measured using a microphone in the device or in a wearable device (smartphone, smart watch, Google Glass) and a speech classification algorithm.

9. Speech intensity—Fatigue influences speech by causing a decrease in sound pressure level, an increase or decrease in the articulation rate and accuracy time of speech (i.e., slower speaking, having longer pauses, making more errors). In addition, it also causes changes in the temporal distribution of acoustic energy. This can be measured using a microphone in the Minicare H-2000 device or in a wearable device (smartphone, smart watch, Google Glass) and a sound intensity classification algorithm.

10. Speech error frequency—When an individual is tired or stressed they often make errors when speaking more frequently. This can be measured using a microphone in the device or in a wearable device (smartphone, smart watch, Google Glass) and a speech classification algorithm.

11. Typing error frequency—A more fatigued patient will make more typing errors when filling out the daily cancer questionnaire on the device. This can be measured by implementing a typing error classification algorithm on the device which analyzes the keyboard input of the cancer patient when they complete the questionnaire each day.

12. Swiping time (when electronically unlocking the device)—A more fatigued patient will have a slower swiping pattern and is more likely to make repeated errors when unlocking the device because of impaired focusing and mental fog. This can be measured using a timer on the device.

The “hard” data (physiological data) can be obtained using the proposed device, preferably with an additional microphone and video camera, as well as with other wearable and/non-wearable devices, in the following ways:

1. Activity counts—Low levels of activity are associated with increased fatigue. This can be measured using accelerometers in wearable devices (e.g., smart patch device, smart watch, smart phone or Google Glass), along with a motion classification algorithm.

2. Anti-oxidant (e.g., Vitamin C or E) conc. —Low levels of anti-oxidants such as vitamin C (i.e., ascorbic acid), Vitamin E (a-Tocopherol), coenzyme Q (Ubiquinol), carotenes, etc. are associated with increased stress and fatigue levels. Using a built-in blood analysis apparatus of the device it is possible to determine the blood concentrations of various antioxidants.

3. Breathing rate—The respiratory rate increases with increasing fatigue level of an individual and in cancer patients is a common symptom in people with cancer during the final days or weeks of life. This can be measured using accelerometers in wearable devices (e.g., smart patch device, smart watch, smart phone or Google Glass), along with a motion classification algorithm.

4. Cortisol level—Cortisol is a steroid hormone released by the adrenal gland metabolic which triggers mechanisms leading to production of compounds used as energy sources in emergency conditions. Cortisol is a validated marker for stress. An increased blood cortisol concentration (of up to 64%) has been reported in all chemotherapy patients. Moreover, there is a link between endogenous cortisol level in predicting acute and delayed nausea during chemotherapy. Using a built-in blood analysis apparatus of the device it is possible to measure the cortisol blood concentration level.

5. Melatonin level—Melatonin is a hormone, produced by the pineal gland, which regulates the body's sleep-wake cycle. Melatonin levels fluctuate throughout the day. Using a built-in blood analysis apparatus of the device it is possible to measure the melatonin blood plasma concentration level.

6. Galvanic skin response (GSR)—Increased GSR is associated with increased stress and fatigue. This can be measured using skin electrodes in a smart watch or other wearable device.

7. Resting Heart Rate (HR)—Elevated HR may be associated with anemia. HR can be measured using various contactless and contact methods, including a vital signs camera, as well as green photoplethysmogram (PPG) and accelerometer sensors integrated in a smart watch or smart patch.

8. Resting Heart Variability (HRV) index—Decreased HRV is linked to increased fatigue. HRV can be measured using various methods, including a vital signs camera, as well as green PPG and accelerometer sensors integrated in a smart watch or smart patch.

9. Red blood cell (RBC) count—Low red blood cell count is associated with anemia. Using a built-in blood analysis apparatus of the device it is possible to determine the RBC count whenever the patient analyzes their blood.

10. Skin temperature—Skin temperature increases with increasing fatigue level due thermal dysregulation (fever). This can be measured using a temperature sensor integrated in a smart watch worn around the patient's wrist.

11. Systolic blood pressure (BP)—Systolic BP increases with increasing stress and fatigue. This can be measured using the pulse arrival time obtained with the green PPG sensor in the smart watch. It can also be obtained from electrocardiogram (ECG) or BP cuff measurements.

An objective fatigue severity score may be determined in another embodiment during a therapy and after the completion of the therapy, which may be a chemotherapy, radiation therapy or other mode of therapy such as radiation therapy, from the combination of the ‘soft’ and ‘hard’ data as exemplified below in Table 1. In this exemplary embodiment, for each ‘soft’ or ‘hard’ parameter, the deviation with respect to a desired ‘normal’ range is scored on a scale from 0-3 and at the end all points are summed to arrive at a CRF severity score. An example scoring system could be: very mild CRF (0-20 points), mild CRF (21-35 points), moderate CRF (36-50 points), and severe CRF (>50 points). Furthermore, the ‘normal’ values/ranges for all parameters (e.g., red blood cell count) may be influenced by such factors as gender and BMI, as well as by patient specific factors, which means that a baseline should be established, by for instance taking measurements before the start of cancer therapy or based on previous cancer therapy cycles. The score can be augmented by additional inputs, based on measurements that are performed at the hospital, for instance during regular outpatient visits. The weighting of each parameter is based on the patient's medical history, their current health state, the temporal rate of variation of the parameter (i.e., does it vary on an hour to hour basis or day to day or week to week) and on the relative importance of the parameter to the clinical diagnosis of fatigue.

Table 1 shows an example of how the various parameters can be weighted and combined to generate a fatigue severity score. It is important to note that the ‘normal’ values/ranges for all parameters (e.g., RBC count) may be influenced by such factors as gender and BMI, as well as by patient specific factors, which means that a baseline should be established.

TABLE 1

| | CRF severity scoring | Weight | 0 normal′ | 1 | 2 | 3 |
|---|---|---|---|---|---|---|
| ‘Hard’ | Activity counts [counts per day] | 1.0 | >900 | 600-899 | 300-599 | <300 |
| parameters | Anti-oxidant conc. [μM/L] | | | | | |
| | Vitamin C | 1.0 | 50-60 | 40-50 | 30-40 | <30 |
| | Vitamin E | 1.0 | 10-40 | 8-10 | 5-8 | <5 |
| | Breathing rate [bpm] | 0.5 | ≤25 | 26-29 | 30-35 | >35 |
| | Cortisol level [mcg/dL] | 1.0 | 3-23 | 23-30 | 30-35 | >35 |
| | GSR [kΩ] | 1.0 | 100-200 | 200-250 | 250-300 | >300 |
| | Resting HR [bpm] | 1.0 | 60-70 | 70-80 | 90-100 | >100 |
| | Resting HRV index [-] | 1.0 | 80-90 | 70-80 | 60-70 | <60 |
| | Melatonin concentration | 1.0 | Daytime | 3.5-6.0 | 6.0-9.0 | >9.0 |
| | [pg/ml] | | 1.9-3.5 | | | |
| | RBC count | 1.0 | 4.7-6.4 | 4.0-4.7 | 3.5-4.0 | <3.5 |
| | [×10⁶ cells/mcL] | | | | | |
| | Skin temperature [° C.] | 0.5 | 34-36 | 33-34 | 32-33 | <32 |
| | Systolic BP [mmHg] | 1.0 | 120-130 | 130-135 | 135-140 | >140 |
| ‘Soft’ | Eating frequency [tpd] | 0.5 | 3-5 | 2-3 | 1-2 | ≤1 |
| parameters | Exercise/intensive activity | 1.0 | 3 | 2 | 1 | 0 |
| | frequency [tpd] | | | | | |
| | Eye jitter[drifts/min] | 0.5 | <5 | 5-10 | 10-15 | >15 |
| | Indecision time [s] | 0.5 | <5 | 5-10 | 10-20 | >20 |
| | Postural sway [body shakes or | 0.5 | <3 | 3-6 | 6-9 | >9 |
| | sways/min] | | | | | |
| | Resting eye movements [blinks | 1.0 | 10-20 | 20-30 | 30-40 | >40 |
| | per min] | | | | | |
| | Sleep disturbance [delta waves | 1.0 | 2-4 | 1-2 | 0.5-1 | <0.5 |
| | per sec] | | | | | |
| | Speaking frequency of negative | 0.5 | 0-1 | 1-5 | 5-10 | >10 |
| | or tired words [wpm] | | | | | |
| | Speech errors [errors/min] | 0.5 | ≤1 | 1-5 | 5-10 | >10 |
| | Speech intensity [dB] | 1.0 | 70-80 | 65-70 | 60-65 | <60 |
| | Swiping time [s] | 0.5 | <2 | 2-5 | 5-10 | >10 |
| | Typing errors [errors/min] | 0.5 | 1 | 1-5 | 5-10 | >10 |

wherein: BP=blood pressure; bpm=breaths/beats per minute; dB=decibels; deg.=degrees; GSR=galvanic skin response; HR=heart rate; HRV=heart rate variability; mcL=microliter; mcg=microgram; pg=pictogram; RBC=red blood cell; tpd=time(s) per day; wpm=words per minute.

Based on the obtained fatigue severity score, appropriate and personalized clinical intervention can be undertaken to improve the management of fatigue and prevent unnecessary hospitalization. This may involve a home visit by a nurse or general practitioner, scheduling of an outpatient visit and hospitalization (if required). In addition, the fatigue severity score may be used to assist in determining whether cancer patients are ready for their next round of therapy and to detect fatigue trends which are useful for forecasting. Additionally, based on the data obtained, a personalized program can be devised to support the cancer patient in various ways, including nutrition advice (including appropriate supplements), relaxation routines and/or targeted exercise routines aimed at fighting fatigue, nausea, muscle mass reduction, bone density reduction, depression.

A number of studies have shown that being active in general and following targeted exercises, e.g., building muscle strength in particular helps to prevent depression and boosts the general feeling of wellness in cancer patients. This comprehensive support package will have both physical and mental benefits beyond fatigue, since nausea and hair loss are also commonly associated with depression in cancer-therapy patients.

In still another embodiment the analyzer 21 is configured to additionally use chronobiology information related to the chronobiology of the person for detecting fatigue. Taking into account the interrelationship between biomarkers of fatigue an algorithm may be used to predict the fatigue level of the patient from diagnosis, through the chemo/radio therapy period as well as during the post-monitoring period. This personalized fatigue score for patients will be based on objective measurements of a number of several key parameters which are highly specific to fatigue, e.g. to cancer related fatigue (CRF), because they are related to the immune and metabolic systems of the body. In a particular implementation an apparatus of the type as shown in FIG. 2 may be used for obtaining these parameters. In an embodiment the following parameters may be used:
1. WBC (White Blood Cell) count, CRP (C-Reactive Protein);
2. HG (hemoglobin), red blood cell count related to anemia;
3. T (Temperature);
4. AO (Anti-oxidants): Non-targeted tissues, such as muscle, are severely affected by oxidative stress during chemotherapy, leading to toxicity and debilitating muscle weakness;
5. ΔC (change in cortisol concentration) and or other immune system parameters such as CRP;
6. ΔM (change in melatonin concentration);
7. CR (circadian Rhythm), which represents the effect of the “chronicity” of cancer treatment (see below for description and relevance of the concept).

An exemplary algorithm for processing these parameters may be:

F(t)=g*CR+Σ((a*WBC)+(b*HG)+(c*T)+(d*AO)+(e*ΔC)+(f*ΔM))

wherein F is the time varying personalized fatigue score and t is time after receiving chemo-/radiation therapy. The coefficients (depicted by a −g) represent the “weight” of each parameter. t<0.0 would refer to time before the treatment session and values of the above parameters for t<0.0 can act as the personalized baseline levels.

The normal range for each parameter is known through clinical data. Three of the above parameters (WBC, HG and T) are efficient parameters for assessing the severity of the effect of cancer treatment on the immune system. Cortisol and melatonin are interrelated validated biomarkers the fluctuations of which during a 24 hour period indicates the health of the HPA axis with significant influence on fatigue level and sleep quality.

Clinically accepted threshold levels for each parameter combined with statistical analysis of the existing data provides trends for fluctuations in these parameters that can be used to determine the “weight” of the parameter represented by coefficients in the formula shown above.

The CR (Circadian Rhythm) represents the influence of chronotoxicity of cancer treatment, which is determined by the biological clock of the patient at the cellular level. This concept defines the toxic effect of cancer treatment drugs on healthy cells which in turn is determined by the “time of day” chosen for cancer treatment.

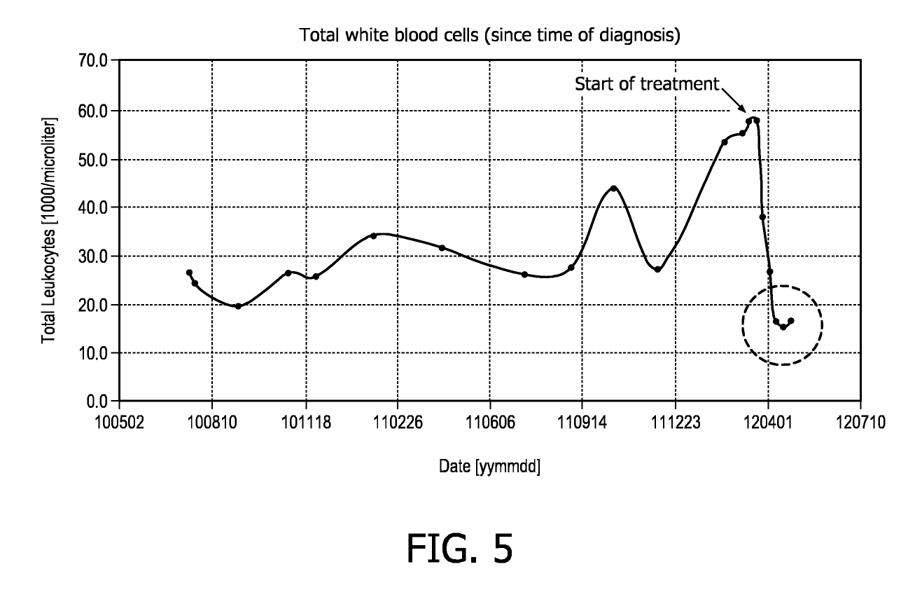

FIG. 4 shows a diagram illustrating the interrelation of the parameters in the fatigue formula and their link to the chronicity of cancer treatment.

The cancer related fatigue score may be translated into a severity ranking indicating whether the fatigue state is severe, mild or low. Each one of these levels can be used to provide specific recommendations for the patient e.g. nutrition or exercise advice, sleeping or activity recommendations, physician consultation or hospitalization. Furthermore, alert messages can be generated when some critical values are detected. This tool will empower both the clinicians, caregivers and the patients as suggested.

In particular, physicians may be supported in taking clinical decisions, i.e. scheduling the time of next treatment session based on personalized data, advice on seeking timely medical intervention to prevent life threatening side effects such as neutropenia. Further, patients (and/or care givers) may be supported in managing their daily lives enabling them to plan and/or adjust events based on the activity level required and their expected fatigue level leading to having more control over their quality of life, which can also help diminishing anxiety and psychological disturbances associated with CRF.

Additionally, the survival rate of cancer patients is correlated with the diurnal cortisol and melatonin. The diurnal cortisol rhythm has been observed to be an independent prognostic factor, as early mortality was associated with ‘flat’ diurnal cortisol rhythms. Moreover, cortisol and melatonin measurements may be performed with current data indicating that they can be used as predictive and prognostic biomarkers of cancer disease.

The circadian rhythm and chronicity of cancer treatment shall be briefly explained in the following. The human metabolism is regulated by the human being's internal clock, i.e. the circadian rhythm (24 h-25 h). The circadian rhythm is a system synchronizing all the biological systems of the human body and any disruption of this system alters the mental, physical, biological functions and immune system. The suprachiasmatic nuclei (SCN) center in the brain is the circadian rhythm center which controls the heart rate, temperature, rest-activity and cortisol/melatonin secretion.

Each of them contributes in a different degree and in order to achieve equilibrium; these factors can be included into clusters instead of targeting each factor one by one. A deregulation of this system leads to insomnia, stress and sleep disorders culminating into fatigue. In addition, the circadian rhythm disruption induces tumor genesis, stress, and down regulates the defense and repair mechanisms of the human body.

Most or all these factors finally contribute to increasing the side-effects and minimizing the efficiency of chemotherapy treatment leading to a decreased quality of life.

Cancer patients have a number of symptoms related to their disease and treatment, such as pain, fatigue, circadian and sleep disturbances that patients, caregivers and clinicians have to manage. Besides that, different types of cancer lead to different needs at different stages of the disease management.

Patients demonstrate sleep disturbances in a different degree especially for early stage breast cancer when they are expecting to undergo surgery or neo-/adjuvant chemotherapy. Moreover, in lung cancer circadian and sleep disturbances are again observed to be in different degree deregulated in early stage and advance stage. The circadian rhythm disruption and sleep disturbances and, consequently, suppression of the immune system have been found to correlate with cancer biology.

Moreover, lower morning energy is associated with higher fatigue. Radiation induced fatigue is higher in patients receiving higher doses of radiation. However, it was observed that fatigue scores were lower in the morning compared to the evening.

Muscle fatigue is muscle specific and involves the loss of muscle function, divided into two components: muscle fatigue and muscle weakness. Monitoring antioxidants can provide data on muscle fatigue as oxidative stress, mediated by cancer or chemotherapeutic agents, is an underlying mechanism of the drug-induced toxicity. Chemotherapy-induced oxidative stress in cancer patients is a reflection of the elevated muscle-derived oxidants, an underlying mechanism for the muscle weakness experienced by patients.

Circulating biomarkers for oxidants serve as an index for the level of oxidative stress in the body and could signify an elevation in muscle derived oxidants. In skeletal muscle, exposure to elevated oxidants are known to cause muscle weakness and accelerate the rate of fatigue on the other hand Antioxidant exposure delays the rate of fatigue, supporting this connection.

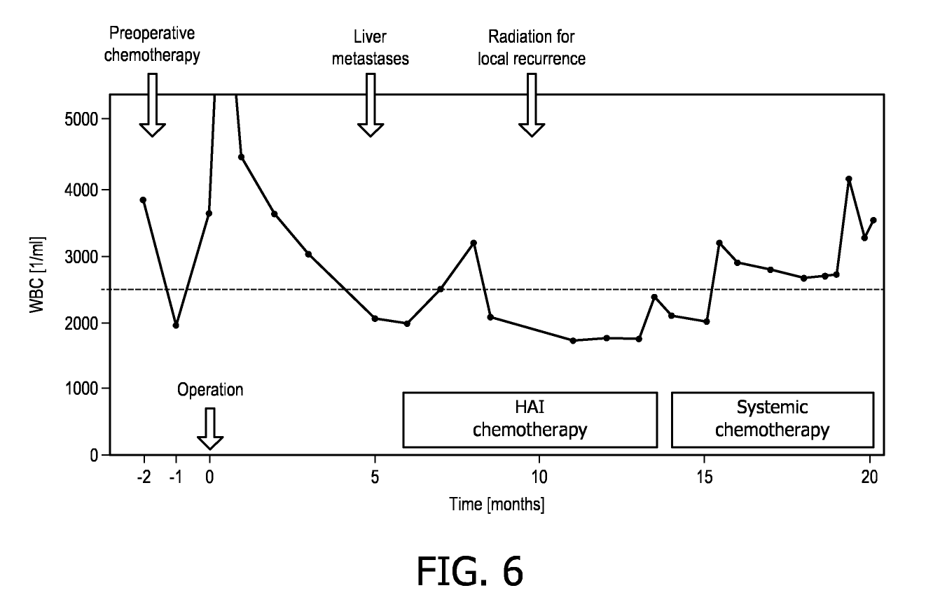

FIGS. 5 to 12 show graphs of various parameters illustrating the influence of fatigue.

FIG. 5 shows the total white blood cell count since the start of a diagnosis and after the start of a treatment.

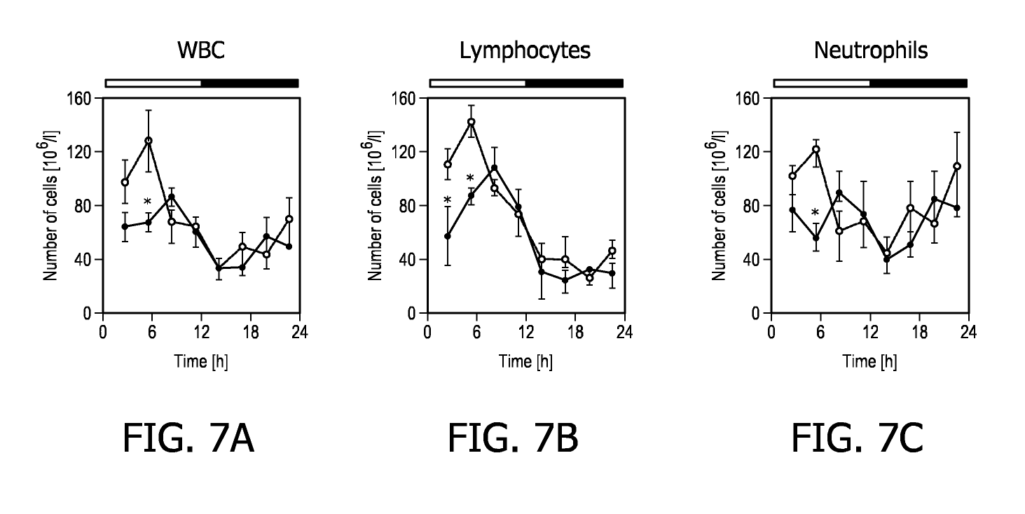

FIG. 6 shows the WBC as influenced by a hepatic arterial infusion chemotherapy for post-operative liver metastases from pancreatic cancer in a patient with leukocytopenia.

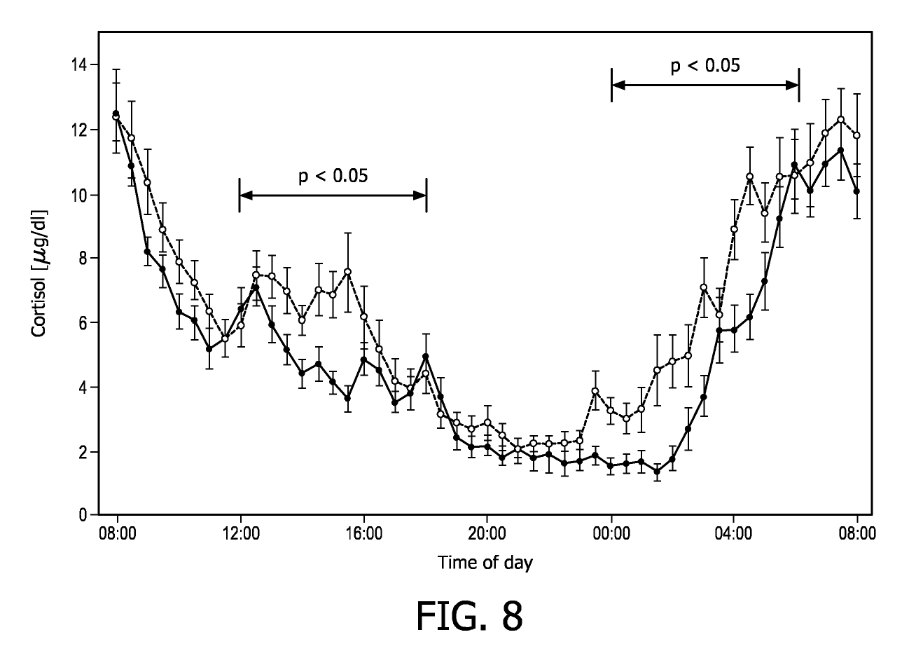

FIG. 7 shows circadian variations in peripheral circulating leukocytes in Clock mutant mice. FIG. 7A shows the total number of white blood cells (WBC), FIG. 7B shows the number of lymphocytes, FIG. 7C shows the number of neutrophils. The open and filled circles are values from wild-type and Clock mutant mice, respectively. Open and solid bars indicate lights on and off, respectively.

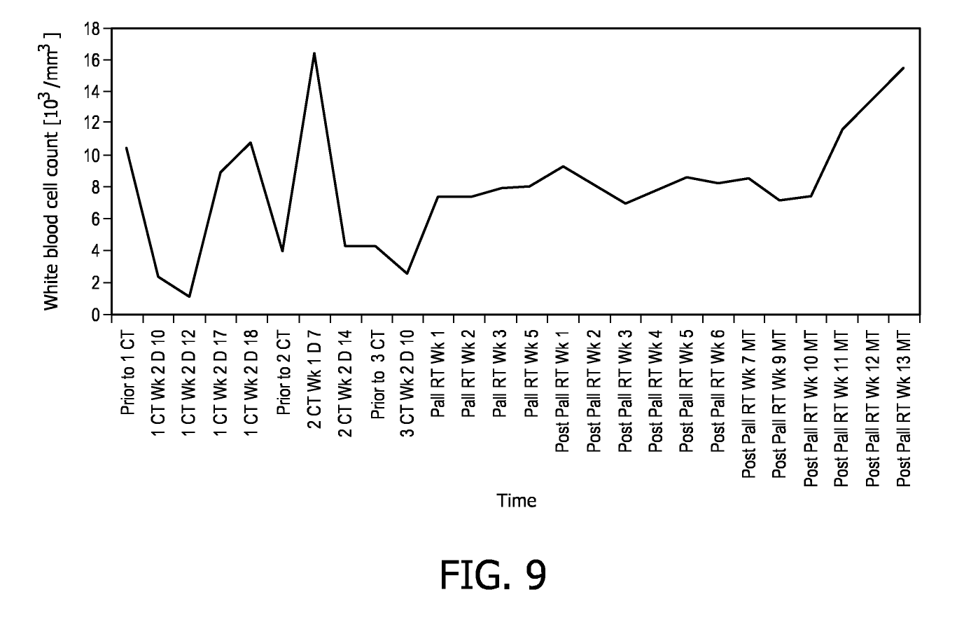

FIG. 8 shows the typical cortisol concentration in serum and saliva and illustrates the circadian effect.

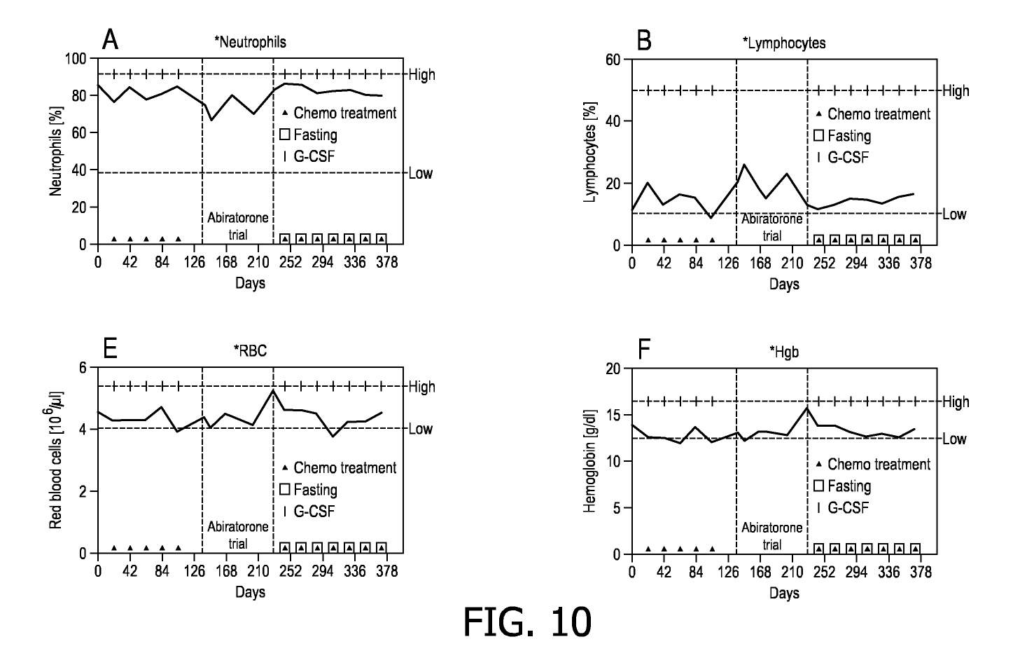

FIG. 9 shows the white blood cell count during cancer treatment course. Hereby mean: 1 CT—First chemotherapy cycle; 2 CT—Second chemotherapy cycle; 3 CT—Third chemotherapy cycle; Wk—Week; D—Day; Pall RT—Palliative radiation therapy; MT—Metronomic therapy.

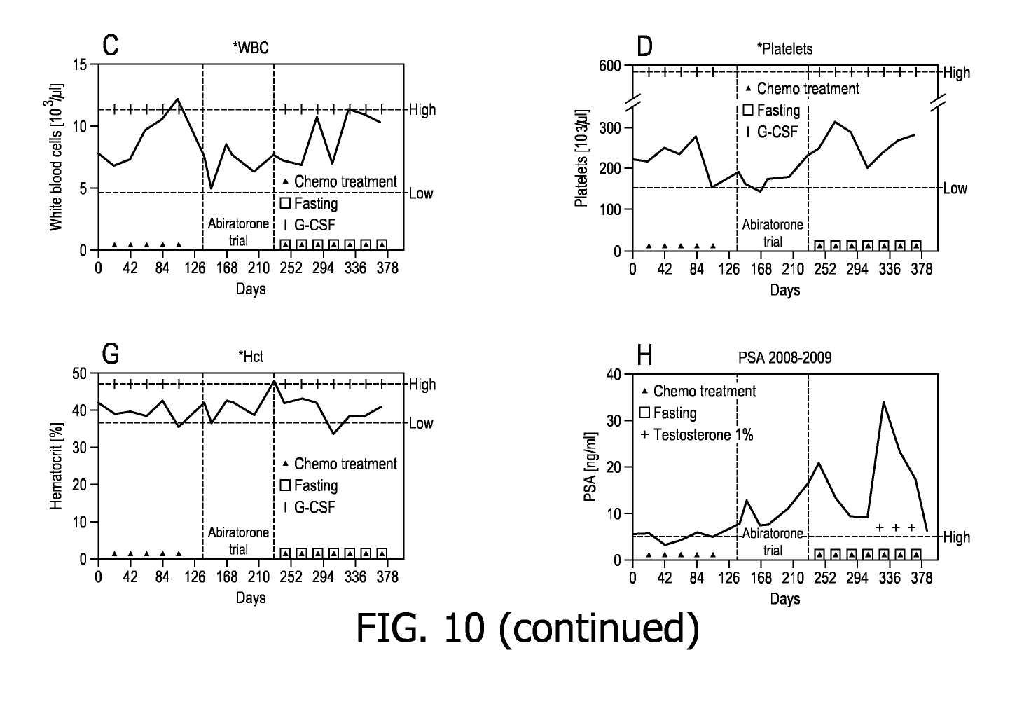

FIG. 10 shows laboratory values of blood cell counts for case 3: (A) Neutrophils; (B) Lymphocytes; (C) White blood cells, WBC; (D) Platelets; (E) Red blood cells, RBC (F) Hemoglobin, Hgb; (G) Hematocrit, Hct; (H) Prostate specific antigen (PSA) level. The patient was enrolled in abiraterone acetate (CYP17 inhibitor) trial for 90 days indicated by vertical dash lines. The patient also received G-CSF (Neulasta) on the day of chemotherapy except during the treatment with abiraterone acetate. A filled triangle indicates a day of chemotherapy; an open square indicates fasting; an arrow indicates testosterone application (cream 1%). Normal ranges of laboratory values are indicated by horizontal dash lines.

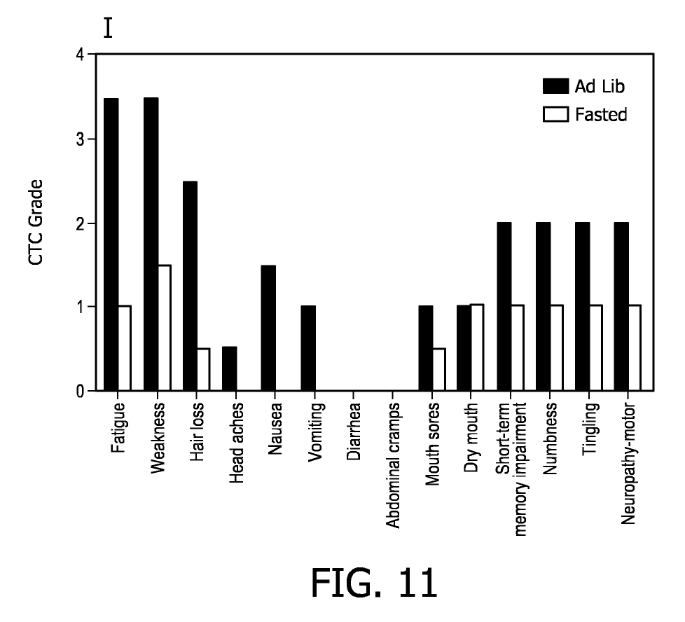

FIG. 11 shows self-reported side-effects after chemotherapy for case 3. The data represent the average of 5 cycles of chemo-alone versus the average of 7 cycles of chemo-fasting treatments.

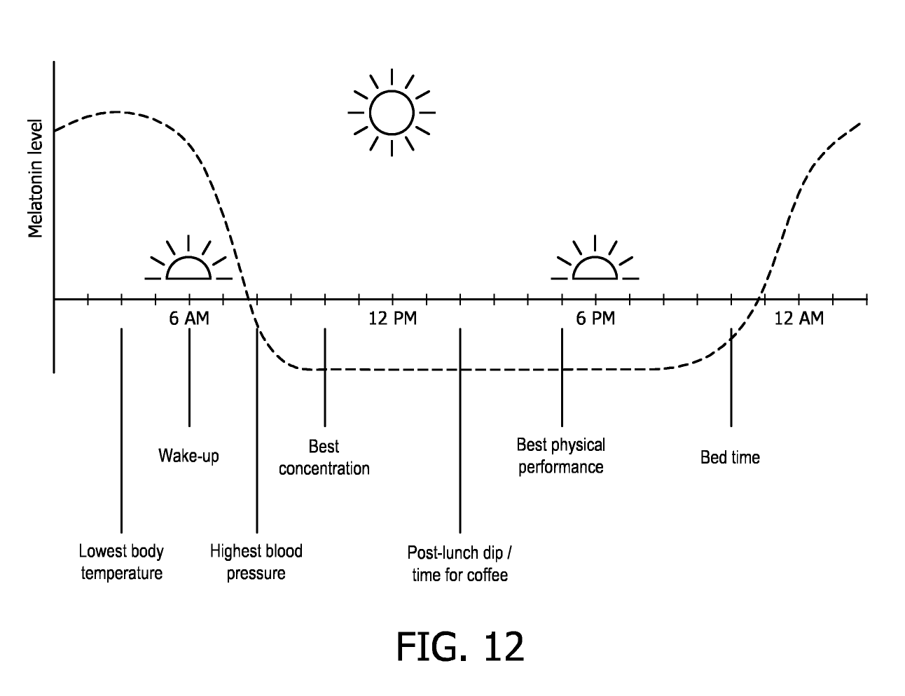

FIG. 12 shows the course of the melatonin level over the course of a day.

The disclosed invention can be used in the disease management of patients during and after treatment for cancer as well as treatment for chronic inflammatory conditions (e.g., rheumatoid arthritis, chronic fatigue syndrome, inflammatory multiple sclerosis, primary Sjögren's syndrome and Systemic lupus erythematosus) and non-inflammatory conditions (e.g., Parkinson's disease, non-inflammatory chronic fatigue syndrome, etc.), in which fatigue has been identified as one of the symptoms of the disease and/or side effects of the treatment.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

A computer program may be stored/distributed on a suitable non-transitory medium, such as an optical storage medium or a solid-state medium supplied together with or as part of other hardware, but may also be distributed in other forms, such as via the Internet or other wired or wireless telecommunication systems.

Any reference signs in the claims should not be construed as limiting the scope.

1. Device for detecting illness- and/or therapy-related fatigue of a person, said device comprising:
an input unit configured to obtain white blood cell count data related to the person's white blood cell count, hemoglobin level data related to the person's hemoglobin level and cortisol level data related to the person's cortisol level, and
an analyzer configured to detect illness- and/or therapy-related fatigue of the person based on the obtained white blood cell count data, hemoglobin level data and cortisol level data, and to determine a fatigue level and to monitor the fatigue level over time.

2. Device as claimed in claim 1,
wherein said analyzer is configured to monitor trends over time in at least one, preferably all, of the obtained white blood cell count data, hemoglobin level data and cortisol level data.

3. Device as claimed in claim 1,
further comprising an interface configured to issue fatigue information, user information, therapy recommendations and/or decision support if fatigue is detected.

4. (canceled)

5. Device as claimed in claim 1,
further comprising an interface configured to issue fatigue information if a fatigue level above a predetermined and/or person-related fatigue level threshold is detected.

6. Device as claimed in claim 1,
wherein said analyzer is configured to additionally use chronobiology information related to the chronobiology of the person for detecting fatigue.

7. Device as claimed in claim 1,
wherein said input unit is configured to obtain person activity data related to one or more activities of the person,
wherein said analyzer is configured to additionally use the obtained person activity data for detecting fatigue.

8. Device as claimed in claim 7,
wherein said input unit is configured to obtain person activity data including one or more of eating habits, exercise frequency, activity level, sleep disturbance, speech pattern, eye movement and body posture.

9. Device as claimed in claim 1,
wherein said input unit is configured to obtain physiological data related to one or more physiological parameters of the person,
wherein said analyzer is configured to additionally use the obtained physiological data for detecting fatigue.

10. Device as claimed in claim 9,
wherein said input unit is configured to obtain physiological data including one or more of biomarker data from blood or other biomaterials such as saliva, urine, tear fluid or hair, melatonin concentration, red blood cell count, anti-oxidant concentration in blood, vital sign measurements such as blood pressure, heart rate, respiratory rate or skin conductance.

11. Device as claimed in claim 1,
wherein said analyzer is configured to determine for the obtained data the respective deviation from a predetermined range, in particular a person-related range, for combining, in particular adding, said deviations and for detecting fatigue, in particular a fatigue level, based on the combined deviations.

12. Method for detecting illness- and/or therapy-related fatigue of a person, said method comprising:
obtaining white blood cell count data related to the person's white blood cell count, hemoglobin level data related to the person's hemoglobin level and cortisol level data related to the person's cortisol level, and
detecting illness- and/or therapy-related fatigue of the person by an analyzer based on the obtained white blood cell count data, hemoglobin level data and cortisol level data.

13. System for detecting illness- and/or therapy-related fatigue of a person, said system comprising:
a white blood cell counter configured to count the white blood cells of the person,
a hemoglobin level sensor configured to determine the hemoglobin level of the person,
a cortisol level sensor configured to determine the cortisol level of the person,
a device as claimed in claim 1 configured to detect illness- and/or therapy-related fatigue of the person based on the data obtained from the white blood cell counter, the hemoglobin level sensor and the cortisol level sensor.

14. System as claimed in claim 13,
further comprising one or more of a video camera, a microphone, a body wearable sensor and a stationary sensor configured to obtain person activity related to one or more activities of the person and/or physiological data related to one or more physiological parameters of the person.

15. Computer program comprising program code means for causing a computer to carry out the steps of the method as claimed in claim 12 when said computer program is carried out on the computer.
